(19) United States
(12) Patent Application Publication
(10) Pub. No.: US 2023/0127084 A1
(43) Pub. Date: Apr. 27, 2023

(54) METHOD AND APPARATUS FOR PERFORMANCE IMPROVEMENT

(71) Applicant: AT&T Intellectual Property I, L.P., Atlanta, GA (US)

(72) Inventors: James H. Pratt, Round Rock, TX (US); Eric Zavesky, Austin, TX (US)

(73) Assignee: AT&T Intellectual Property I, L.P., Atlanta, GA (US)

(21) Appl. No.: 17/511,952

(22) Filed: Oct. 27, 2021

(51) Int. Cl. G06Q 10/06; G06Q 10/10; G06F 3/01

(52) U.S. Cl. CPC G06Q 10/06393; G06Q 10/1093; G06F 3/016; G06F 3/011

(57) ABSTRACT

Aspects of the subject disclosure may include, for example, a method operating at a processing system including a processor, including detecting a difference between current biometric information of a user and target biometric information of the user exceeding a threshold at a performance event of the user to identify a biometric deficiency, detecting an absence of a desired environmental component for the user at the performance event, selecting a remediation experience responsive to the detecting the biometric deficiency and the detecting the absence of the desired environmental component, facilitating presentation of the remediation experience to the user at the performance event to generate a modified performance by the user, and adjusting the presentation of the remediation experience to the user at a termination of the performance event. Other embodiments are disclosed.

### FIELD OF THE DISCLOSURE

The subject disclosure relates to a method and apparatus for performance improvement.

### BACKGROUND

Modern telecommunications systems provide consumers with telephony capabilities while accessing a large variety of content. Consumers are no longer bound to specific locations when communicating with others or when enjoying multimedia content or accessing the varied resources available via the Internet. Network capabilities have expanded and have created additional interconnections and new opportunities for using mobile communication devices in a variety of situations. Intelligent devices offer new means for experiencing network interactions in ways that anticipate consumer desires and provide solutions to problems.

### BRIEF DESCRIPTION OF THE DRAWINGS

Reference will now be made to the accompanying drawings, which are not necessarily drawn to scale, and wherein:

FIG. 1 is a block diagram illustrating an exemplary, non-limiting embodiment of a communications network in accordance with various aspects described herein.

FIG. 2A is a block diagram illustrating an example, non-limiting embodiment of a system functioning within the communication network of FIG. 1 to provide a remediation experience for improving performance of a user in accordance with various aspects described herein.

FIG. 2B is a workflow diagram illustrating an example, non-limiting embodiment of a system functioning within the communication network of FIG. 1 and the system of FIG. 2A to provide a remediation experience to improve performance in accordance with various aspects described herein.

FIG. 2C depict illustrative embodiments of methods to provide a remediation experience to improve performance in accordance with various aspects described herein.

FIG. 3 is a block diagram illustrating an example, non-limiting embodiment of a virtualized communication network in accordance with various aspects described herein.

FIG. 4 is a block diagram of an example, non-limiting embodiment of a computing environment in accordance with various aspects described herein.

FIG. 5 is a block diagram of an example, non-limiting embodiment of a mobile network platform in accordance with various aspects described herein.

FIG. 6 is a block diagram of an example, non-limiting embodiment of a communication device in accordance with various aspects described herein.

### DETAILED DESCRIPTION

The subject disclosure describes, among other things, illustrative embodiments for providing a remediation experience to improve performance. A performance event can be identified for a user. A biometric deficiency can be identified based on a difference between current biometric information of the user with target biometric information. An absence of a desired environmental component can be determined for the user during the performance event. A remediation experience can be selected based on the biometric deficiency or the absence of the desired environmental component. The remediation experience can be presented to the user to generate a modified user performance. Other embodiments are described in the subject disclosure.

One or more aspects of the subject disclosure include a device, including a processing system including a processor and a memory that stores executable instructions that, when executed by the processing system, facilitate performance of operations. The operations can include determining a performance event of a user. The operations can also include detecting a difference between current biometric information of the user and target biometric information of the user exceeding a threshold at the performance event. The operations can further include detecting an absence of a desired environmental component for the user at the performance event. The operations can include selecting a remediation experience responsive to the detecting the disharmony and the detecting the absence of the desired environmental component. The operations can also include facilitating presentation of the remediation experience to the user at the performance event to generate a modified performance by the user. The operations can further include monitoring the modified performance by the user responsive to the facilitating the presentation of the remediation experience to the user at the performance event, and, in turn, adjusting the presentation of the remediation experience to the user at the performance event according to the monitoring the performance of the user. The operations can include detecting a termination of the performance event, and, in turn, adjusting the presentation of the remediation experience to the user responsive to detecting the termination of the performance event.

One or more aspects of the subject disclosure include a non-transitory machine-readable medium, comprising executable instructions that, when executed by a processing system including a processor, facilitate performance of operations. The operations can include detecting a difference between current biometric information of a user and target biometric information of the user exceeding a threshold at a performance event of the user. The operations can also include detecting an absence of a desired environmental component for the user at the performance event. The operations can further include selecting a remediation experience responsive to the detecting the disharmony or the detecting the absence of the desired environmental component. The operations can include facilitating presentation of the remediation experience to the user at the performance event to generate a modified performance by the user. The operations can also include monitoring the modified performance by the user responsive to the facilitating the presentation of the remediation experience to the user at the performance event, and, in turn, adjusting the presentation of the remediation experience to the user at the performance event according to the monitoring the performance of the user. The operations can further include adjusting the presentation of the remediation experience to the user at a termination of the performance event.

One or more aspects of the subject disclosure include a method performed by a processing system including a processor. The method can include detecting, by a processing system including a processor, a difference between current biometric information of a user and target biometric information of the user exceeding a threshold at a performance event of the user. The method can also include detecting, by the processing system, an absence of a desired environmental component for the user at the performance event. The method can further include selecting, by the processing system, a remediation experience responsive to the detecting the disharmony or the detecting the absence of the desired environmental component. The method can include facilitating, by the processing system, presentation of the remediation experience to the user at the performance event to generate a modified performance by the user. The method can also include adjusting, by the processing system, the presentation of the remediation experience to the user at a termination of the performance event.

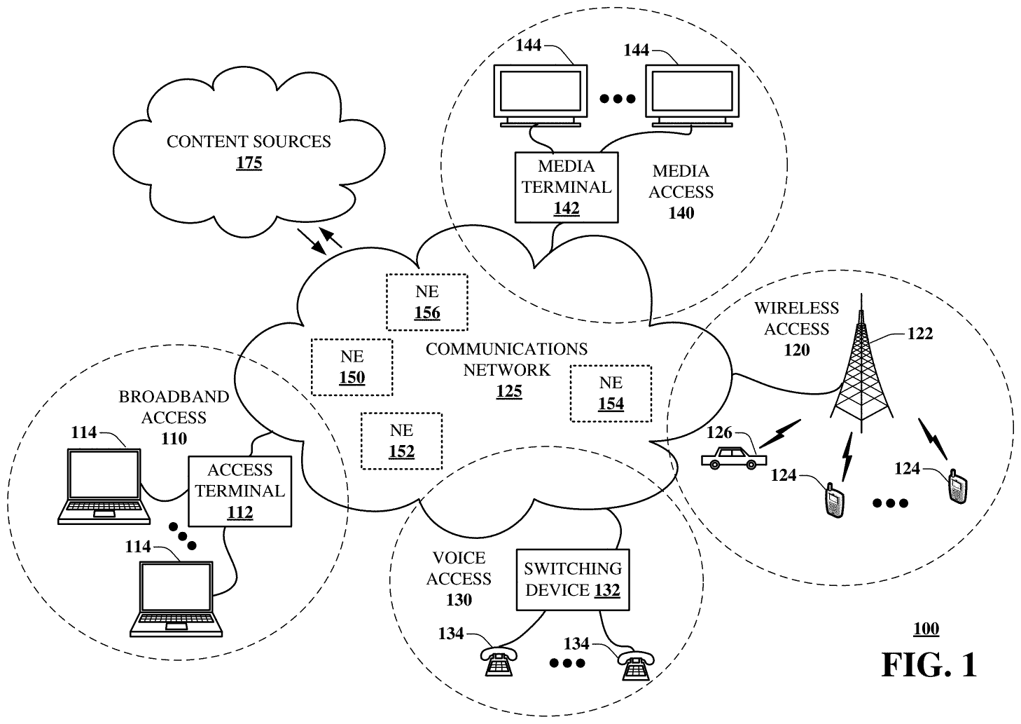

Referring now to FIG. 1, a block diagram is shown illustrating an example, non-limiting embodiment of a system 100 in accordance with various aspects described herein. For example, system 100 can facilitate in whole or in part for providing a remediation experience to improve performance. A performance event can be identified for a user. A biometric deficiency can be identified based on a difference between current biometric information of the user with target biometric information. An absence of a desired environmental component can be determined for the user during the performance event. A remediation experience can be selected based on the biometric deficiency or the absence of the desired environmental component. The remediation experience can be presented to the user to generate a modified user performance. In particular, a communications network 125 is presented for providing broadband access 110 to a plurality of data terminals 114 via access terminal 112, wireless access 120 to a plurality of mobile devices 124 and vehicle 126 via base station or access point 122, voice access 130 to a plurality of telephony devices 134, via switching device 132 and/or media access 140 to a plurality of audio/video display devices 144 via media terminal 142. In addition, communication network 125 is coupled to one or more content sources 175 of audio, video, graphics, text and/or other media. While broadband access 110, wireless access 120, voice access 130 and media access 140 are shown separately, one or more of these forms of access can be combined to provide multiple access services to a single client device (e.g., mobile devices 124 can receive media content via media terminal 142, data terminal 114 can be provided voice access via switching device 132, and so on).

The communications network 125 includes a plurality of network elements (NE) 150, 152, 154, 156, etc. for facilitating the broadband access 110, wireless access 120, voice access 130, media access 140 and/or the distribution of content from content sources 175. The communications network 125 can include a circuit switched or packet switched network, a voice over Internet protocol (VoIP) network, Internet protocol (IP) network, a cable network, a passive or active optical network, a 4G, 5G, or higher generation wireless access network, WIMAX network, UltraWideband network, personal area network or other wireless access network, a broadcast satellite network and/or other communications network.

In various embodiments, the access terminal 112 can include a digital subscriber line access multiplexer (DSLAM), cable modem termination system (CMTS), optical line terminal (OLT) and/or other access terminal. The data terminals 114 can include personal computers, laptop computers, netbook computers, tablets or other computing devices along with digital subscriber line (DSL) modems, data over coax service interface specification (DOCSIS) modems or other cable modems, a wireless modem such as a 4G, 5G, or higher generation modem, an optical modem and/or other access devices.

In various embodiments, the base station or access point 122 can include a 4G, 5G, or higher generation base station, an access point that operates via an 802.11 standard such as 802.11n, 802.11ac or other wireless access terminal. The mobile devices 124 can include mobile phones, e-readers, tablets, phablets, wireless modems, and/or other mobile computing devices.

In various embodiments, the switching device 132 can include a private branch exchange or central office switch, a media services gateway, VoIP gateway or other gateway device and/or other switching device. The telephony devices 134 can include traditional telephones (with or without a terminal adapter), VoIP telephones and/or other telephony devices.

In various embodiments, the media terminal 142 can include a cable head-end or other TV head-end, a satellite receiver, gateway or other media terminal 142. The display devices 144 can include televisions with or without a set top box, personal computers and/or other display devices.

In various embodiments, the content sources 175 include broadcast television and radio sources, video on demand platforms and streaming video and audio services platforms, one or more content data networks, data servers, web servers and other content servers, and/or other sources of media.

In various embodiments, the communications network 125 can include wired, optical and/or wireless links and the network elements 150, 152, 154, 156, etc. can include service switching points, signal transfer points, service control points, network gateways, media distribution hubs, servers, firewalls, routers, edge devices, switches and other network nodes for routing and controlling communications traffic over wired, optical and wireless links as part of the Internet and other public networks as well as one or more private networks, for managing subscriber access, for billing and network management and for supporting other network functions.

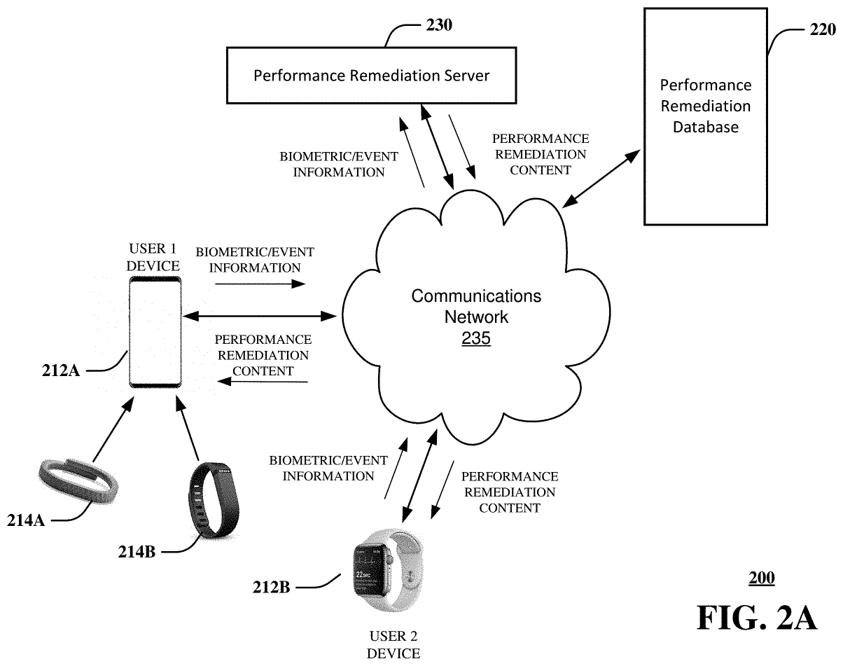

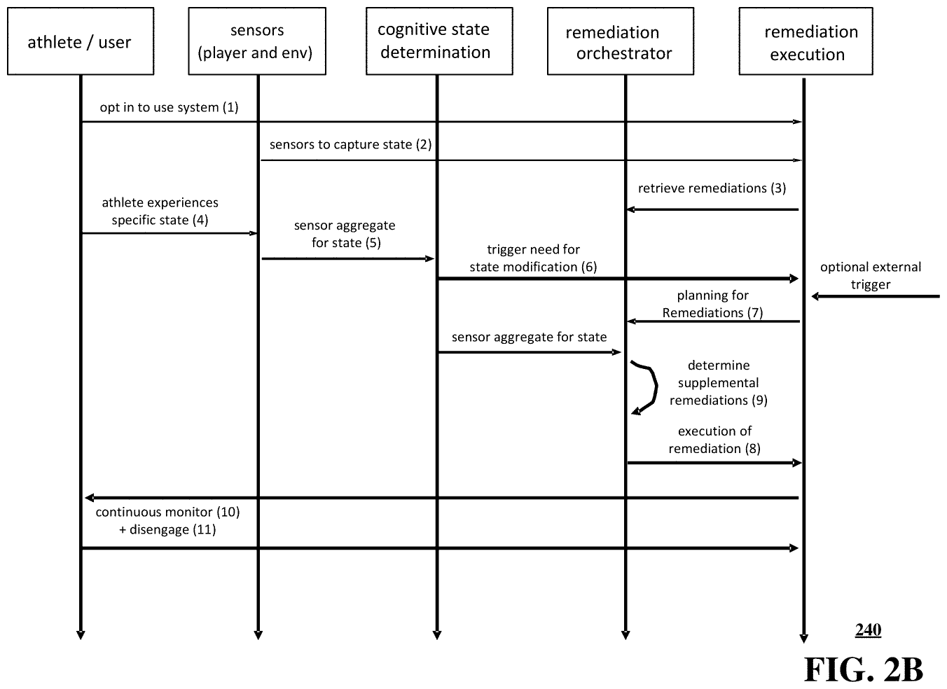

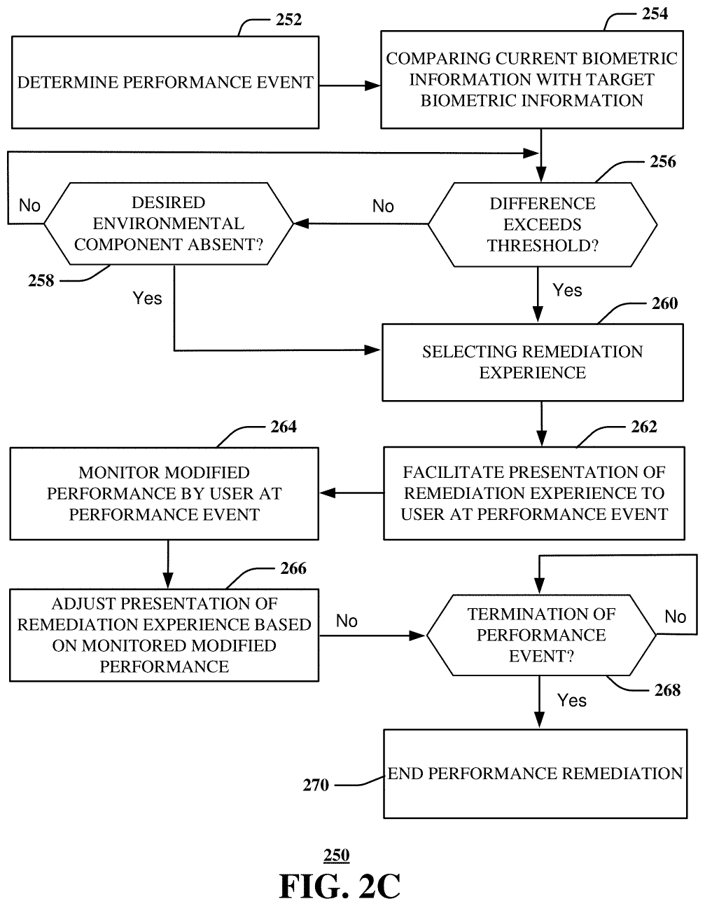

FIG. 2A is a block diagram illustrating an example, non-limiting embodiment of a system functioning within the communication network of FIG. 1 in accordance with various aspects described herein. For example, system 200 can facilitate in whole or in part for providing a remediation experience to improve performance. A performance event can be identified for a user. A biometric deficiency can be identified based on a difference between current biometric information of the user with target biometric information. An absence of a desired environmental component can be determined for the user during the performance event. A remediation experience can be selected based on the biometric deficiency or the absence of the desired environmental component. The remediation experience can be presented to the user to generate a modified user performance.

In one or more embodiments, the system 200 can include a communications network 235 communicatively coupled to devices 212A-B. The devices 212A-B can belong or be assigned to users. The devices 212A-B can access communications and other services from the communications network 235 via, for example, wireless communications links, such as cellular links. In one or more embodiments, devices 212A-B can capture information about the user including biometric information describing the current biometric state of the user. For example, a device 212A, such as a smart phone of user 1, can capture biometric information from the body of user 1 via one or more wearable devices 214A-B that are in communication with the device 212A. In another example, device 212B, such as a smart watch of user 2, can capture biometric information directly via its contact with the skin of user 2 and can receive additional information, indirectly, from other wearable devices on the body of user 2. The devices 212A-B can pass the biometric information to the communication network 235. In one or more embodiments, the devices 212A-B can share information with the communication network 235 and/or receive information from the communication network 235 regarding upcoming or currently occurring performance events, such as athletic contests or training sessions in which the user of the device 212A is a participant. In one or more embodiments, the devices 212A-B can receive remediation content information from the communications network 235. This remediation content can be presented via the devices 212A-B to modify the users' performance during performance events, such as athletic contests or training sessions.

In one or more embodiments, the system 200 can include a performance remediation server 230 and a performance remediation database 220 communicatively coupled to the communications network 235. The performance remediation server 230 can receive biometric information from user devices 212A-B. The performance remediation server 230 can also receive information regarding an upcoming or currently occurring performance event, such as an athletic contest or training session. The performance remediation server 230 can assess the biometric information to determine if a particular user is physiologically and/or emotionally operating within a target range that is associated with optimal and/or normalized performance for the performance event.

In one or more embodiments, biometric information received from device 212A of user 1 might indicate a particular set of metrics that are consisted with an elevated level of stress occurring in the body of user 1. Further, the performance remediation server 230 can access historical data for user 1 from a performance remediation database 220. This historical data can associate user 1 biometric information with user 1 event performance. For example, the historical data can indicate that user 1 exhibits sub optimal performance in an athletic event, such as a bicycling race, during times of elevated stress levels. Alternatively, athletic performance generally improves at lower stress levels and peaks when the stress level is in a target range.

In one or more embodiments, performance event information received from device 212A can be used by the performance remediation server 230 to determine the type and character of the performance event. For example, user 1 might be currently engaged in an actual competition, a training session, a warm-up period, a warm down period, or a time of rest and recovery. Each of these types of “performance” are important to the overall performance and well-being of user 1. By correctly assessing the current performance event, the performance remediation server 230 can compare incoming biometric information to historical biometric information of the correct type, such that, for example, biometric information during a current racing session is selectively compared to biometric information from a past racing session or a past training session, while biometric information from a rest and recovery session is compared to a past rest and recovery session. The performance remediation server 230 can analyze incoming biometric and event information with historical biometric and event information to assess the current performance condition of user 1 against historical benchmarks and targets and can then add this current information to historical information in the performance remediation database to continue to build and model the performance of user 1.

In one or more embodiments, the performance remediation server 230 can provide performance remediation content to the communications network 235, which can be directed to user devices 212A-B for presentation to users at those devices. The performance remediation server 230 can access the performance remediation content from the performance remediation database 220, where the performance remediation content can be associated with biometric information and performance event information of users. For example, user 2 may routinely train for an athletic competition while listening to a particular set of music. The performance remediation database 220 can store this historical association along with biometric information from these training sessions. Subsequently, if the performance remediation server 230 detects that user 2 is engaged in the same performance event (e.g., a training session) or a similar performance event, then the performance remediation server 230 can access the associated performance remediation content (e.g., the set of music) and provide this content to the device 212B of user 2, so that device 212B can present this content to user 2 during the performance event.

In one or more embodiments, the performance remediation server 230 can determine, from a user's current biometric information and the performance event information, whether to provide performance remediation content and, if so, then which performance remediation content to provide to augment or improve the user's performance. If, for example, user 2 can be currently exhibiting biometric information within a target range or currently exhibiting optimal performance, while simultaneously receiving no performance remediation content. In this case, the performance remediation server 230 may determine to continue this situation (e.g., provide no performance remediation content because it is not needed). Alternatively, user can be currently exhibiting out-of-target biometric information and/or sub optimal performance, while simultaneously receiving no performance remediation content. In this case, the performance remediation server 230 may determine to alter this situation by providing performance remediation content, as indicated by the performance remediation database 220, to improve performance, because the data indicates that it is needed.

The popularity and intensity of competition in professional sports and other athletic areas continues to increase over time. This growth appears to coincide (or perhaps cause) the level of distractions at sporting events to increase over time. These distractions, both audio-based (e.g., jeering, airhorns, clackers) and visual-based (e.g., signs, balloons, digital jumbotron displays) have become more numerous and can cause additional stresses for players or participants in these performance events.

It is found that, first, although not typically used during athletic contests, simple biometrics (e.g., pulse, salinity, oxygen intake) can now assess the stress/mental state of a player. These systems could be added to detect disharmony between a player's peak performance state and their current performance state. Second, remediation content can be generalized (e.g., brown noise, calming sounds) or specific (e.g., visual appearances of a family member in the crowd, a cloudy day, or a sunset on the horizon). If a disharmony in biometric state and/or sub-optional performance state is detected, then appropriate remediation content can be selected and made available to the player to restore their performance capabilities. Third, augmented reality techniques are found to be growing in efficiency and impact in the virtual reality space. Similarly, a personalized environment can be selected and made available to a player to improve their performance. Presenting remediation content can improve athletic performance with minimal disruption for other players, attendees, and viewers.

In one or more embodiments, remediation content or motivational augmentation can be used to improve athletic performance. The performance remediation server 230 can detect disharmony between a user's current biometric/emotional state and their target biometric/emotional state (e.g., excited, relaxed, etc.) that is known to be a good state for a performance context (e.g., final pass, dash, etc.). The performance remediation server 230 can access known remediation content/techniques (e.g., hearing a crowd's cheering sound) that can be presented to the user to modify their biometric/emotional state from their current state to their target based on historical data, individual preferences, and novel situations. In one or more embodiments, the remediation content can be used to purposefully diminish the user's reality to improve performance. For example, the presentation of audio remediation content, such as peaceful sounds that mimic the user's training environment could be used to diminish the effect of distracting or discourage crowd noises. In one or more embodiments, the audio remediation content can remove an entire channel of stimulus from the user's environment. For example, the system 200 can selectively remove all audio from the user's experience by using a noise cancellation technique when the system 200 detects that such remediation is needed. The performance remediation server 230 can instruct the user's device 212A to sample the noise environment and provide cancellation audio to the user to achieve noise cancellation. In one or more embodiments, the system 200 can perform selective noise cancellation, where, for example unwanted noise sources are diminished and/or cancelled, while desired noise sources are allowed to continue and/or amplified. For example, the performance remediation server 230 can instruct the user's device 212A to perform generalized noise cancellation for environmental audio, while the performance remediation server 230 provides an augmented signal of one participant in the athletic event, such as a coach's voice or a quarterback's voice.

The remediation content can also be used to augment the user's performance reality. For example, the presentation of audio remediation content to add crowd cheering can compensate for a performance event environment that does not include fans (e.g., a location where fans are not permitted due to a pandemic). The remediation content can thereby purposefully diminish or augment reality, while not creating virtual reality immersion to adapt the user's perceived environment and move their biometric/emotional state toward their target state (relaxation, excitement). The remediation content can present the user with a combination of audio and video modifications or additions.

In one or more embodiments, the performance remediation server 230 can detect opportunities for providing augmented or diminished reality to a user athlete (or other performer) during a performance event. These opportunities can be detected based biometric information captured from the user, contextual information captured from the performance environment, and actual performance information. When an opportunity for performance mediation (augmented or diminished) is identified, the performance remediation server 230 can select a proper performance remediation content based on user history, user preference, and/or best practices. The performance remediation content can be provided to a user device 212A for presentation to the user during a selected context of the performance event. For example, the performance remediation content can be provided during warm-up period, competition, warm down, timeout, and/or rest and recovery. The presentation can include audio and/or video elements. The presentation can include haptic elements, such as vibration or other movement, or environmental elements, such as heating or cooling. The performance remediation content can be present via any type of user device and/or via the devices of other users detected to be in close proximity to the user and/or environmentally available devices, such as public video displays and/or sound systems. The performance event information, including biometric information, performance information, context information, performance remediation content selection, and monitored outcomes can be stored as historical information for future analysis and sharing with others.

In one or more embodiments, the system 200 can be used by athletes in training. The diminished or augmented reality created by presentation of the performance remediation content can modify the athlete's focus or perception of reality in ways that aid in performance. Complications in the field of performance, other players, crowd noise, and/or visual distractions can be diminished by changing the athlete's focus and/or diminishing their importance to the athlete's field of hearing or vision. For example, during an athletic contest away from the athlete's “home field,” the performance remediation content can use a diminish algorithm to convert the “away field” soundscape into something that sounds more like the home field environment. For example, the system 200 can detect biometric markers using sensors worn by the athlete. A high heart rate, perhaps a rate that is not consistent with the athlete's current performance effort, can indicate that the athlete is emotionally stressed. The system 200 can respond by providing remediation content that alters the athlete's noise spectrum and calms the athlete's emotions.

FIG. 2B is a workflow diagram illustrating an example, non-limiting embodiment of a system functioning within the communication network of FIG. 1 and the system 200 of FIG. 2A to provide a remediation experience to improve performance in accordance with various aspects described herein. In one or more embodiments, a user or athlete can register to use the system, in step 1. The user can be preregistered to the system with an option to opt-in or opt-out.

In one or more embodiments, the system 200 can capture and log user biometric information from sensors on the user during training sessions, in step 2 . For example, the user can wear biometric sensors that provide information to communication devices on the user or proximate to the user. The system 200 can capture sensor information from the performance environment of the user. For example, the system 200 can capture data including temperature, weather, background noise, and/or light intensity. The system 200 can collect performance statistical information that is germane to the type of training and/or competition. For example, if the user is an endurance athlete, the system 200 can collect and store distance, timing, and/or results information. If the user is engaged in a competitive team sport, such as baseball, the system 200 can collect and store relevant batting, pitching, fielding, and baserunning statistics. The system 200 can store the user's biometric, environmental, and performance information in a database, such that it can be analyzed, correlated, and used for making decisions about future remediation actions.

In one or more embodiments, the system 200 can create a profile for the user or link to an existing profile from the user, in step 3. The system 200 can base a new user profile on a template profile according to the type of athletic performance that is desired (e.g., endurance athlete, team sport). The user can provide preference information to direct the type of performance remediation provided. For example, the user can provide information on preference for audio, visual, or haptic content. The user can provide favorite training music, locations, and/or images.

In one or more embodiments, the user can enter a performance state known to the system 200, in step 4. For example, the user can enter a performance state of competition or training or rest/recovery. The user's current state can be provided to the system 200 by the user by, for example, entering an application on a communication device 212A. The system 200 can detect the current state by accessing calendar information and/or location information indicating a training session or an athletic contest. Once the system 200 knows the state of the user, it can continue to monitor to determine if the user transitions to a different state (e.g., completes the competition).

In one or more embodiments, the system 200 can collect and aggregate sensory information during the current performance state, in step 5. The system 200 can detect a need for performance remediation, in step 6. The system 200 can compare the current sensory information to target sensory information. The target sensory information can be based on historic sensory information for the user captured in past performance events. The target sensory information can be based on benchmark data. The system 200 can determine that the user could benefit from performance remediation based on the current sensory information substantially differing from the target sensory information. The system 200 can receive the sensory information from a local personal area network of the user or proximate to the user (e.g., excessive sweating).

In one or more embodiments, the system 200 can identify the context of the user's performance event can be identified by the performance remediation. For example, the system 200 can use location information to determine that the user is at a basketball arena and/or calendar information to determine that scheduled basketball game is underway. More precise information, such as timing information can be used by the system 200 to determine that the scheduled basketball game is in a break period, such as half time or a timeout. The system 200 can aggregate the contextual information to determine when to deliver (or not deliver) remediation content to the user.

In one or more embodiments, the system 200 can plan for remediation content, in step 7. The system 200 can analyze the current biometric state and the current performance context to determine an “ideal” remediation content to deliver to the user or to update remediation content that is already being delivered to the user. The system 200 can determine if there a missing component in the user's performance environment. For example, an athlete may be accustomed to loud crowd noise during competition and have a demonstrated record of better athletic performance in the presence of loud crowd noise. The system 200 can determine from sensory data (e.g., audio from a microphone) that the performance environment is very quiet, and, therefore, this component is missing from the athlete's environment. The system 200 can determine to remediate this issue by selecting crowd noise as a component of the performance remediation content.

In one or more embodiments, the system 200 can execute the performance remediation, in step 8. The system 200 can send performance remediation content to a user device 212A of the athlete, where the device can present the content for the user to experience. For example, the system 200 can send crowd noise to the user device 212A, which is reproduced by an audio system for the athlete to hear. The presentation of the performance remediation content can be performed by an aural headset or glasses worn by the athlete. The performance remediation content can include video content that can be used to diminish or augment the athlete's reality during the performance event. For example, video can be overlayed onto a see-through aperture in a pair of goggles, a windshield, a visor, or a pair of glasses. The system 200 can provide the performance remediation content to an audio/visual system at a stadium, such that the content is reproduced and presented via the stadium playback system.

In one or more embodiments, the system 200 can monitor for changes in the biometric information and/or aggregated state of the athlete after the initiation of presentation of performance remediation content. The system 200 can analyze these changes (or, conversely, the absence of changes) to determine whether to modify the performance remediation content, in step 9. The system 200 can monitor for changes in the performance event context to determine if the athlete has transitioned from one state to another state. For example, the performance state can change from active competition to rest/recovery at the end of the competition. The system 200 can automatically stop further performance remediation, in step 10, based on the change in performance state. The system 200 can determine, at any point, that the athlete has disabled further performance remediation and can disengage further remediation, in step 11.

In one or more embodiments, the system 200 can improve athletic performance by providing motivational sounds, such as cheering, at the right time and context, during a competitive/game situation. The system 200 can provide visual motivation, at the right time and context, to diminish performance distractions and/or augment performance reality. The system 200 can provide performance appropriate cheering (or booing) during competition to motivate performance. The system 200 can create an optimal atmosphere for sports performance using sounds and visuals and modify neurochemical responses to environment utilizing augmented reality.

In one or more embodiments, the system 200 can provide an oversight and/or override function for use by competition organizers, referees, and/or coaches. The oversight/override function can allow for selectively disabling the system 200 or monitoring the use of the system 200 to ensure the performance remediation is performed in a manner that is consistent with the rules of the contest and that does not render an “unfair advantage” within the spirit of the competition. For example, the oversight/override function can enable remediation during certain times of the contest, but not others, or can only enable certain types of remediations, but not others. The oversight/override function can provide collect information that can be used for further training the system 200 and/or for further studying performance of athletes.

In one or more embodiments, the system 200 can utilize AI/Machine learning to analyze historical data, such as biometric information, performance information, and performance remediation information, to guide decision making. Historical information for a registered user and for other users can be mined for patterns and techniques for improving performance.

FIG. 2C depicts an illustrative embodiment of a method in accordance with various aspects described herein. The method 250 can be performed by the system 200 of FIG. 2B, the communication system 100 of FIG. 2A, or a combination thereof. In step 252, the current occurrence of a performance event can be determined. For example, location and calendar data can be used to determine that a user is currently engaged in an athletic contest, a training session, or a pre-event or post-event circumstance. In step 254, current biometric information captured at the user can be compared to target biometric information derived from historical performance data. In step 256, if a difference between the current biometric information and the target biometric data exceeds a threshold, then a biometric deficiency can be identified. An absence from the performance environment of a desired component can be detected, in step 258. The detection of an identified biometric deficiency and/or an absence of a desired environmental component, in steps 256 and 258, can be an indication that the user is likely to be performing in a sub optimal way and can result in the selection of a remediation experience or content, in step 260.

In step 262, presentation of the performance remediation content or experience can be facilitated by, for example, reproducing the remediation content at a device of the user. Modification of the user's performance under the influence of the performance remediation content can be monitored, in step 264. The performance remediation experience can be adjusted, in step 266, based on the monitored user performance, by adjusting the remediation content and/or the presentation of the content to the user. Termination of the performance event can be detected, in step 268, and, if terminated, then the performance remediation can be ended, in step 270.

While for purposes of simplicity of explanation, the respective processes are shown and described as a series of blocks in FIG. 2C, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein.

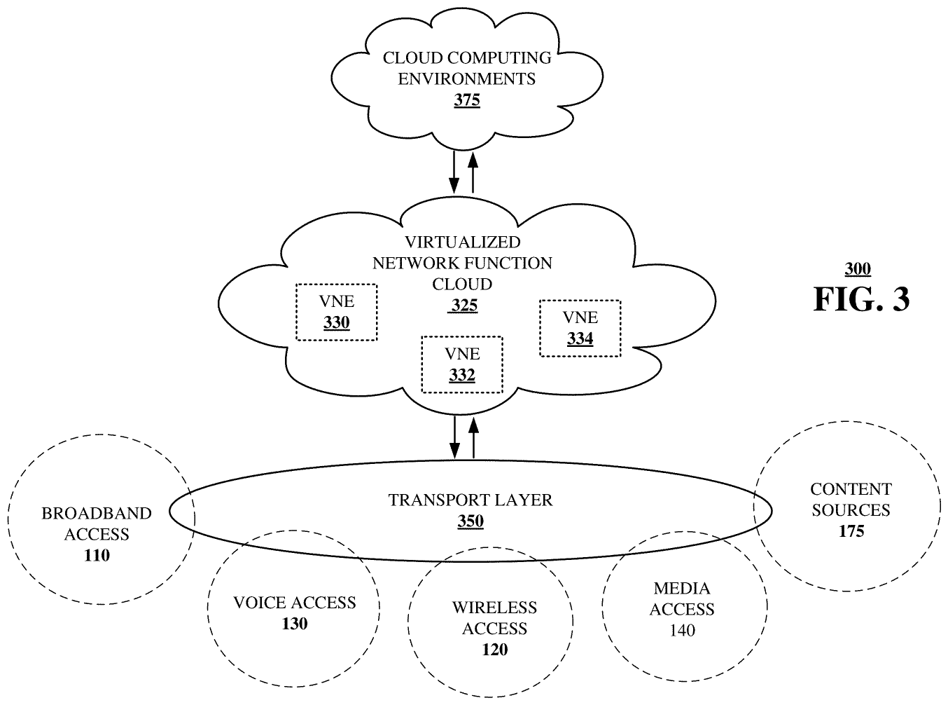

Referring now to FIG. 3, a block diagram 300 is shown illustrating an example, non-limiting embodiment of a virtualized communication network in accordance with various aspects described herein. In particular a virtualized communication network is presented that can be used to implement some or all of the subsystems and functions of system 100, the subsystems and functions of system 200, and method 230 presented in FIGS. 1, 2A, 2B, 2C, and 3. For example, virtualized communication network 300 can facilitate in whole or in part for providing a remediation experience to improve performance. A performance event can be identified for a user. A biometric deficiency can be identified based on a difference between current biometric information of the user with target biometric information. An absence of a desired environmental component can be determined for the user during the performance event. A remediation experience can be selected based on the biometric deficiency or the absence of the desired environmental component. The remediation experience can be presented to the user to generate a modified user performance.

In particular, a cloud networking architecture is shown that leverages cloud technologies and supports rapid innovation and scalability via a transport layer 350, a virtualized network function cloud 325 and/or one or more cloud computing environments 375. In various embodiments, this cloud networking architecture is an open architecture that leverages application programming interfaces (APIs); reduces complexity from services and operations; supports more nimble business models; and rapidly and seamlessly scales to meet evolving customer requirements including traffic growth, diversity of traffic types, and diversity of performance and reliability expectations.

In contrast to traditional network elements—which are typically integrated to perform a single function, the virtualized communication network employs virtual network elements (VNEs) 330, 332, 334, etc. that perform some or all of the functions of network elements 150, 152, 154, 156, etc. For example, the network architecture can provide a substrate of networking capability, often called Network Function Virtualization Infrastructure (NFVI) or simply infrastructure that is capable of being directed with software and Software Defined Networking (SDN) protocols to perform a broad variety of network functions and services. This infrastructure can include several types of substrates. The most typical type of substrate being servers that support Network Function Virtualization (NFV), followed by packet forwarding capabilities based on generic computing resources, with specialized network technologies brought to bear when general-purpose processors or general-purpose integrated circuit devices offered by merchants (referred to herein as merchant silicon) are not appropriate. In this case, communication services can be implemented as cloud-centric workloads.

As an example, a traditional network element 150 (shown in FIG. 1), such as an edge router can be implemented via a VNE 330 composed of NFV software modules, merchant silicon, and associated controllers. The software can be written so that increasing workload consumes incremental resources from a common resource pool, and moreover so that it is elastic: so, the resources are only consumed when needed. In a similar fashion, other network elements such as other routers, switches, edge caches, and middle-boxes are instantiated from the common resource pool. Such sharing of infrastructure across a broad set of uses makes planning and growing infrastructure easier to manage.

In an embodiment, the transport layer 350 includes fiber, cable, wired and/or wireless transport elements, network elements and interfaces to provide broadband access 110, wireless access 120, voice access 130, media access 140 and/or access to content sources 175 for distribution of content to any or all of the access technologies. In particular, in some cases a network element needs to be positioned at a specific place, and this allows for less sharing of common infrastructure. Other times, the network elements have specific physical layer adapters that cannot be abstracted or virtualized and might require special DSP code and analog front ends (AFEs) that do not lend themselves to implementation as VNEs 330, 332 or 334. These network elements can be included in transport layer 350.

The virtualized network function cloud 325 interfaces with the transport layer 350 to provide the VNEs 330, 332, 334, etc. to provide specific NFVs. In particular, the virtualized network function cloud 325 leverages cloud operations, applications, and architectures to support networking workloads. The virtualized network elements 330, 332 and 334 can employ network function software that provides either a one-for-one mapping of traditional network element function or alternately some combination of network functions designed for cloud computing. For example, VNEs 330, 332 and 334 can include route reflectors, domain name system (DNS) servers, and dynamic host configuration protocol (DHCP) servers, system architecture evolution (SAE) and/or mobility management entity (MME) gateways, broadband network gateways, IP edge routers for IP-VPN, Ethernet and other services, load balancers, distributers and other network elements. Because these elements do not typically need to forward large amounts of traffic, their workload can be distributed across a number of servers—each of which adds a portion of the capability, and which creates an elastic function with higher availability overall than its former monolithic version. These virtual network elements 330, 332, 334, etc. can be instantiated and managed using an orchestration approach similar to those used in cloud compute services.

The cloud computing environments 375 can interface with the virtualized network function cloud 325 via APIs that expose functional capabilities of the VNEs 330, 332, 334, etc. to provide the flexible and expanded capabilities to the virtualized network function cloud 325. In particular, network workloads may have applications distributed across the virtualized network function cloud 325 and cloud computing environment 375 and in the commercial cloud or might simply orchestrate workloads supported entirely in NFV infrastructure from these third-party locations.

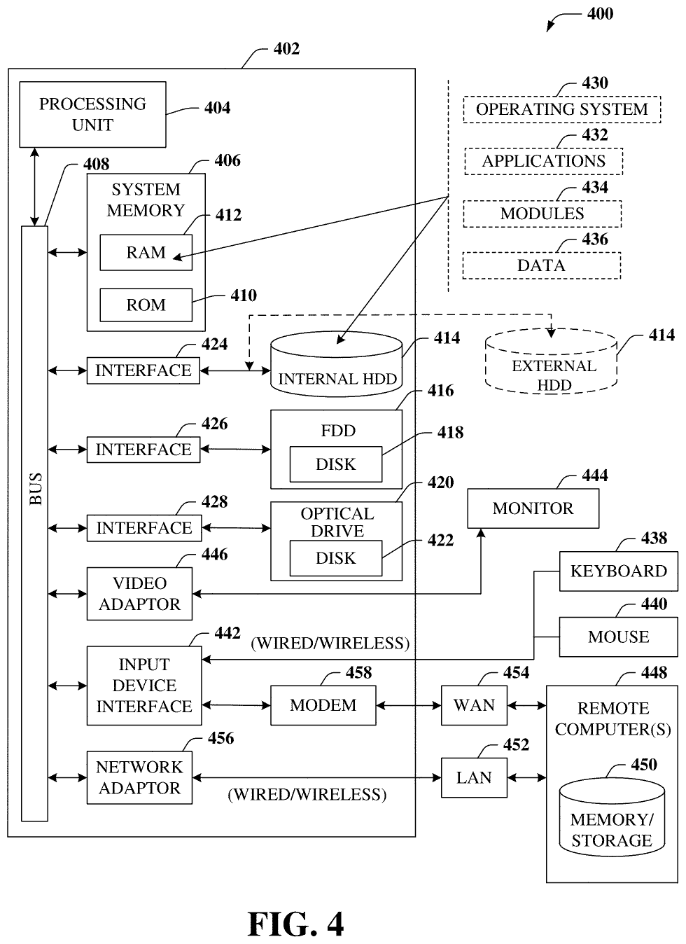

Turning now to FIG. 4, there is illustrated a block diagram of a computing environment in accordance with various aspects described herein. In order to provide additional context for various embodiments of the embodiments described herein, FIG. 4 and the following discussion are intended to provide a brief, general description of a suitable computing environment 400 in which the various embodiments of the subject disclosure can be implemented. In particular, computing environment 400 can be used in the implementation of network elements 150, 152, 154, 156, access terminal 112, base station or access point 122, switching device 132, media terminal 142, and/or VNEs 330, 332, 334, etc. Each of these devices can be implemented via computer-executable instructions that can run on one or more computers, and/or in combination with other program modules and/or as a combination of hardware and software. For example, computing environment 400 can facilitate in whole or in part for providing a remediation experience to improve performance. A performance event can be identified for a user. A biometric deficiency can be identified based on a difference between current biometric information of the user with target biometric information. An absence of a desired environmental component can be determined for the user during the performance event. A remediation experience can be selected based on the biometric deficiency or the absence of the desired environmental component. The remediation experience can be presented to the user to generate a modified user performance.

Generally, program modules comprise routines, programs, components, data structures, etc., that perform particular tasks or implement particular abstract data types.

Moreover, those skilled in the art will appreciate that the methods can be practiced with other computer system configurations, comprising single-processor or multiprocessor computer systems, minicomputers, mainframe computers, as well as personal computers, hand-held computing devices, microprocessor-based or programmable consumer electronics, and the like, each of which can be operatively coupled to one or more associated devices.

As used herein, a processing circuit includes one or more processors as well as other application specific circuits such as an application specific integrated circuit, digital logic circuit, state machine, programmable gate array or other circuit that processes input signals or data and that produces output signals or data in response thereto. It should be noted that while any functions and features described herein in association with the operation of a processor could likewise be performed by a processing circuit.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically comprise a variety of media, which can comprise computer-readable storage media and/or communications media, which two terms are used herein differently from one another as follows. Computer-readable storage media can be any available storage media that can be accessed by the computer and comprises both volatile and nonvolatile media, removable and non-removable media. By way of example, and not limitation, computer-readable storage media can be implemented in connection with any method or technology for storage of information such as computer-readable instructions, program modules, structured data or unstructured data.

Computer-readable storage media can comprise, but are not limited to, random access memory (RAM), read only memory (ROM), electrically erasable programmable read only memory (EEPROM),flash memory or other memory technology, compact disk read only memory (CD-ROM), digital versatile disk (DVD) or other optical disk storage, magnetic cassettes, magnetic tape, magnetic disk storage or other magnetic storage devices or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and comprises any information delivery or transport media. The term “modulated data signal” or signals refers to a signal that has one or more of its characteristics set or changed in such a manner as to encode information in one or more signals. By way of example, and not limitation, communication media comprise wired media, such as a wired network or direct-wired connection, and wireless media such as acoustic, RF, infrared and other wireless media.

With reference again to FIG. 4, the example environment can comprise a computer 402, the computer 402 comprising a processing unit 404, a system memory 406 and a system bus 408. The system bus 408 couples system components including, but not limited to, the system memory 406 to the processing unit 404. The processing unit 404 can be any of various commercially available processors. Dual microprocessors and other multiprocessor architectures can also be employed as the processing unit 404.

The system bus 408 can be any of several types of bus structure that can further interconnect to a memory bus (with or without a memory controller), a peripheral bus, and a local bus using any of a variety of commercially available bus architectures. The system memory 406 comprises ROM 410 and RAM 412. A basic input/output system (BIOS) can be stored in a non-volatile memory such as ROM, erasable programmable read only memory (EPROM), EEPROM, which BIOS contains the basic routines that help to transfer information between elements within the computer 402, such as during startup. The RAM 412 can also comprise a high-speed RAM such as static RAM for caching data.

The computer 402 further comprises an internal hard disk drive (HDD) 414 (e.g., EIDE, SATA), which internal HDD 414 can also be configured for external use in a suitable chassis (not shown), a magnetic floppy disk drive (FDD) 416, (e.g., to read from or write to a removable diskette 418) and an optical disk drive 420, (e.g., reading a CD-ROM disk 422 or, to read from or write to other high capacity optical media such as the DVD). The HDD 414, magnetic FDD 416 and optical disk drive 420 can be connected to the system bus 408 by a hard disk drive interface 424, a magnetic disk drive interface 426 and an optical drive interface 428, respectively. The hard disk drive interface 424 for external drive implementations comprises at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 402, the drives and storage media accommodate the storage of any data in a suitable digital format. Although the description of computer-readable storage media above refers to a hard disk drive (HDD), a removable magnetic diskette, and a removable optical media such as a CD or DVD, it should be appreciated by those skilled in the art that other types of storage media which are readable by a computer, such as zip drives, magnetic cassettes, flash memory cards, cartridges, and the like, can also be used in the example operating environment, and further, that any such storage media can contain computer-executable instructions for performing the methods described herein.

A number of program modules can be stored in the drives and RAM 412, comprising an operating system 430, one or more application programs 432, other program modules 434 and program data 436. All or portions of the operating system, applications, modules, and/or data can also be cached in the RAM 412. The systems and methods described herein can be implemented utilizing various commercially available operating systems or combinations of operating systems.

A user can enter commands and information into the computer 402 through one or more wired/wireless input devices, e.g., a keyboard 438 and a pointing device, such as a mouse 440. Other input devices (not shown) can comprise a microphone, an infrared (IR) remote control, a joystick, a game pad, a stylus pen, touch screen or the like. These and other input devices are often connected to the processing unit 404 through an input device interface 442 that can be coupled to the system bus 408, but can be connected by other interfaces, such as a parallel port, an IEEE 1394 serial port, a game port, a universal serial bus (USB) port, an IR interface, etc.

A monitor 444 or other type of display device can be also connected to the system bus 408 via an interface, such as a video adapter 446. It will also be appreciated that in alternative embodiments, a monitor 444 can also be any display device (e.g., another computer having a display, a smart phone, a tablet computer, etc.) for receiving display information associated with computer 402 via any communication means, including via the Internet and cloud-based networks. In addition to the monitor 444, a computer typically comprises other peripheral output devices (not shown), such as speakers, printers, etc.

The computer 402 can operate in a networked environment using logical connections via wired and/or wireless communications to one or more remote computers, such as a remote computer(s) 448. The remote computer(s) 448 can be a workstation, a server computer, a router, a personal computer, portable computer, microprocessor-based entertainment appliance, a peer device or other common network node, and typically comprises many or all of the elements described relative to the computer 402, although, for purposes of brevity, only a remote memory/storage device 450 is illustrated. The logical connections depicted comprise wired/wireless connectivity to a local area network (LAN) 452 and/or larger networks, e.g., a wide area network (WAN) 454. Such LAN and WAN networking environments are commonplace in offices and companies, and facilitate enterprise-wide computer networks, such as intranets, all of which can connect to a global communications network, e.g., the Internet.

When used in a LAN networking environment, the computer 402 can be connected to the LAN 452 through a wired and/or wireless communication network interface or adapter 456. The adapter 456 can facilitate wired or wireless communication to the LAN 452, which can also comprise a wireless AP disposed thereon for communicating with the adapter 456.

When used in a WAN networking environment, the computer 402 can comprise a modem 458 or can be connected to a communications server on the WAN 454 or has other means for establishing communications over the WAN 454, such as by way of the Internet. The modem 458, which can be internal or external and a wired or wireless device, can be connected to the system bus 408 via the input device interface 442. In a networked environment, program modules depicted relative to the computer 402 or portions thereof, can be stored in the remote memory/storage device 450. It will be appreciated that the network connections shown are example and other means of establishing a communications link between the computers can be used.

The computer 402 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This can comprise Wireless Fidelity (Wi-Fi) and BLUETOOTH® wireless technologies. Thus, the communication can be a predefined structure as with a conventional network or simply an ad hoc communication between at least two devices.

Wi-Fi can allow connection to the Internet from a couch at home, a bed in a hotel room or a conference room at work, without wires. Wi-Fi is a wireless technology similar to that used in a cell phone that enables such devices, e.g., computers, to send and receive data indoors and out anywhere within the range of a base station. Wi-Fi networks use radio technologies called IEEE 802.11 (a, b, g, n, ac, ag, etc.) to provide secure, reliable, fast wireless connectivity. A Wi-Fi network can be used to connect computers to each other, to the Internet, and to wired networks (which can use IEEE 802.3 or Ethernet). Wi-Fi networks operate in the unlicensed 2.4 and 5 GHz radio bands for example or with products that contain both bands (dual band), so the networks can provide real-world performance similar to the basic 10BaseT wired Ethernet networks used in many offices.

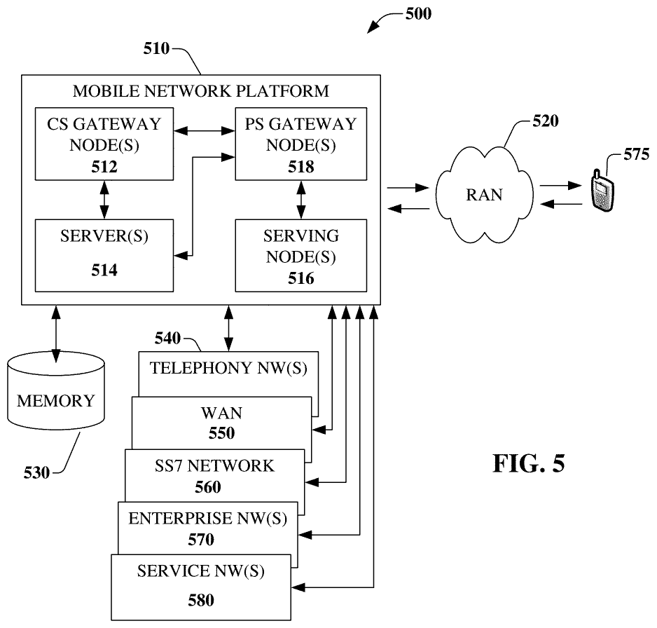

Turning now to FIG. 5, an embodiment 500 of a mobile network platform 510 is shown that is an example of network elements 150, 152, 154, 156, and/or VNEs 330, 332, 334, etc. For example, platform 510 can facilitate in whole or in part for providing a remediation experience to improve performance. A performance event can be identified for a user. A biometric deficiency can be identified based on a difference between current biometric information of the user with target biometric information. An absence of a desired environmental component can be determined for the user during the performance event. A remediation experience can be selected based on the biometric deficiency or the absence of the desired environmental component. The remediation experience can be presented to the user to generate a modified user performance. In one or more embodiments, the mobile network platform 510 can generate and receive signals transmitted and received by base stations or access points such as base station or access point 122. Generally, mobile network platform 510 can comprise components, e.g., nodes, gateways, interfaces, servers, or disparate platforms, that facilitate both packet-switched (PS) (e.g., internet protocol (IP), frame relay, asynchronous transfer mode (ATM)) and circuit-switched (CS) traffic (e.g., voice and data), as well as control generation for networked wireless telecommunication. As a non-limiting example, mobile network platform 510 can be included in telecommunications carrier networks and can be considered carrier-side components as discussed elsewhere herein. Mobile network platform 510 comprises CS gateway node(s) 512 which can interface CS traffic received from legacy networks like telephony network(s) 540 (e.g., public switched telephone network (PSTN), or public land mobile network (PLMN)) or a signaling system #7 (SS7) network 560. CS gateway node(s) 512 can authorize and authenticate traffic (e.g., voice) arising from such networks. Additionally, CS gateway node(s) 512 can access mobility, or roaming, data generated through SS7 network 560; for instance, mobility data stored in a visited location register (VLR), which can reside in memory 530. Moreover, CS gateway node(s) 512 interfaces CS-based traffic and signaling and PS gateway node(s) 518. As an example, in a 3GPP UMTS network, CS gateway node(s) 512 can be realized at least in part in gateway GPRS support node(s) (GGSN). It should be appreciated that functionality and specific operation of CS gateway node(s) 512, PS gateway node(s) 518, and serving node(s) 516, is provided and dictated by radio technology(ies) utilized by mobile network platform 510 for telecommunication over a radio access network 520 with other devices, such as a radiotelephone 575.

In addition to receiving and processing CS-switched traffic and signaling, PS gateway node(s) 518 can authorize and authenticate PS-based data sessions with served mobile devices. Data sessions can comprise traffic, or content(s), exchanged with networks external to the mobile network platform 510, like wide area network(s) (WANs) 550, enterprise network(s) 570, and service network(s) 580, which can be embodied in local area network(s) (LANs), can also be interfaced with mobile network platform 510 through PS gateway node(s) 518. It is to be noted that WANs 550 and enterprise network(s) 570 can embody, at least in part, a service network(s) like IP multimedia subsystem (IMS). Based on radio technology layer(s) available in technology resource(s) or radio access network 520, PS gateway node(s) 518 can generate packet data protocol contexts when a data session is established; other data structures that facilitate routing of packetized data also can be generated. To that end, in an aspect, PS gateway node(s) 518 can comprise a tunnel interface (e.g., tunnel termination gateway (TTG) in 3GPP UMTS network(s) (not shown)) which can facilitate packetized communication with disparate wireless network(s), such as Wi-Fi networks.

In embodiment 500, mobile network platform 510 also comprises serving node(s) 516 that, based upon available radio technology layer(s) within technology resource(s) in the radio access network 520, convey the various packetized flows of data streams received through PS gateway node(s) 518. It is to be noted that for technology resource(s) that rely primarily on CS communication, server node(s) can deliver traffic without reliance on PS gateway node(s) 518; for example, server node(s) can embody at least in part a mobile switching center. As an example, in a 3GPP UMTS network, serving node(s) 516 can be embodied in serving GPRS support node(s) (SGSN).

For radio technologies that exploit packetized communication, server(s) 514 in mobile network platform 510 can execute numerous applications that can generate multiple disparate packetized data streams or flows, and manage (e.g., schedule, queue, format . . . ) such flows. Such application(s) can comprise add-on features to standard services (for example, provisioning, billing, customer support . . . ) provided by mobile network platform 510. Data streams (e.g., content(s) that are part of a voice call or data session) can be conveyed to PS gateway node(s) 518 for authorization/authentication and initiation of a data session, and to serving node(s) 516 for communication thereafter. In addition to application server, server(s) 514 can comprise utility server(s), a utility server can comprise a provisioning server, an operations and maintenance server, a security server that can implement at least in part a certificate authority and firewalls as well as other security mechanisms, and the like. In an aspect, security server(s) secure communication served through mobile network platform 510 to ensure network's operation and data integrity in addition to authorization and authentication procedures that CS gateway node(s) 512 and PS gateway node(s) 518 can enact. Moreover, provisioning server(s) can provision services from external network(s) like networks operated by a disparate service provider; for instance, WAN 550 or Global Positioning System (GPS) network(s) (not shown). Provisioning server(s) can also provision coverage through networks associated to mobile network platform 510 (e.g., deployed and operated by the same service provider), such as the distributed antennas networks shown in FIG. 1(s) that enhance wireless service coverage by providing more network coverage.

It is to be noted that server(s) 514 can comprise one or more processors configured to confer at least in part the functionality of mobile network platform 510. To that end, the one or more processor can execute code instructions stored in memory 530, for example. It should be appreciated that server(s) 514 can comprise a content manager, which operates in substantially the same manner as described hereinbefore.

In example embodiment 500, memory 530 can store information related to operation of mobile network platform 510. Other operational information can comprise provisioning information of mobile devices served through mobile network platform 510, subscriber databases; application intelligence, pricing schemes, e.g., promotional rates, flat-rate programs, couponing campaigns; technical specification(s) consistent with telecommunication protocols for operation of disparate radio, or wireless, technology layers; and so forth. Memory 530 can also store information from at least one of telephony network(s) 540, WAN 550, SS7 network 560, or enterprise network(s) 570. In an aspect, memory 530 can be, for example, accessed as part of a data store component or as a remotely connected memory store.

In order to provide a context for the various aspects of the disclosed subject matter, FIG. 5, and the following discussion, are intended to provide a brief, general description of a suitable environment in which the various aspects of the disclosed subject matter can be implemented. While the subject matter has been described above in the general context of computer-executable instructions of a computer program that runs on a computer and/or computers, those skilled in the art will recognize that the disclosed subject matter also can be implemented in combination with other program modules. Generally, program modules comprise routines, programs, components, data structures, etc. that perform particular tasks and/or implement particular abstract data types.

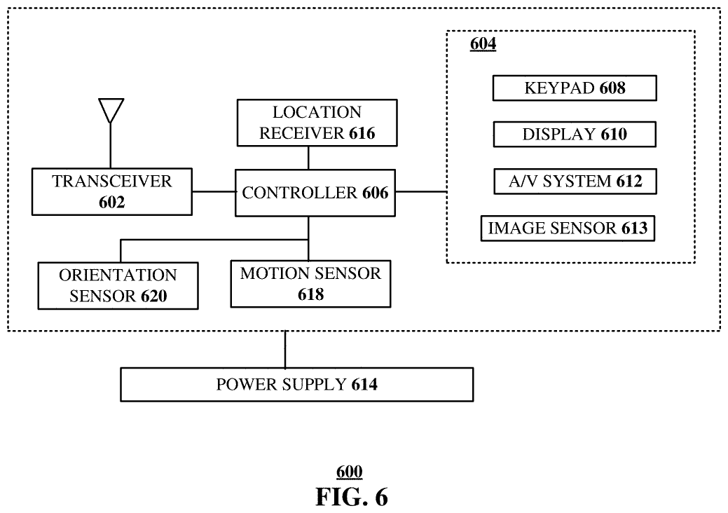

Turning now to FIG. 6, an illustrative embodiment of a communication device 600 is shown. The communication device 600 can serve as an illustrative embodiment of devices such as data terminals 114, mobile devices 124, vehicle 126, display devices 144 or other client devices for communication via either communications network 125. For example, computing device 600 can facilitate in whole or in part for providing a remediation experience to improve performance. A performance event can be identified for a user. A biometric deficiency can be identified based on a difference between current biometric information of the user with target biometric information. An absence of a desired environmental component can be determined for the user during the performance event. A remediation experience can be selected based on the biometric deficiency or the absence of the desired environmental component. The remediation experience can be presented to the user to generate a modified user performance.

The communication device 600 can comprise a wireline and/or wireless transceiver 602 (herein transceiver 602), a user interface (UI) 604, a power supply 614, a location receiver 616, a motion sensor 618, an orientation sensor 620, and a controller 606 for managing operations thereof. The transceiver 602 can support short-range or long-range wireless access technologies such as Bluetooth®, ZigBee®, Wi-Fi, DECT, or cellular communication technologies, just to mention a few (Bluetooth® and ZigBee® are trademarks registered by the Bluetooth® Special Interest Group and the ZigBee® Alliance, respectively). Cellular technologies can include, for example, CDMA-1X, UMTS/HSDPA, GSM/GPRS, TDMA/EDGE, EV/DO, WiMAX, SDR, LTE, as well as other next generation wireless communication technologies as they arise. The transceiver 602 can also be adapted to support circuit-switched wireline access technologies (such as PSTN), packet-switched wireline access technologies (such as TCP/IP, VoIP, etc.), and combinations thereof.

The UI 604 can include a depressible or touch-sensitive keypad 608 with a navigation mechanism such as a roller ball, a joystick, a mouse, or a navigation disk for manipulating operations of the communication device 600. The keypad 608 can be an integral part of a housing assembly of the communication device 600 or an independent device operably coupled thereto by a tethered wireline interface (such as a USB cable) or a wireless interface supporting for example Bluetooth®. The keypad 608 can represent a numeric keypad commonly used by phones, and/or a QWERTY keypad with alphanumeric keys. The UI 604 can further include a display 610 such as monochrome or color LCD (Liquid Crystal Display), OLED (Organic Light Emitting Diode) or other suitable display technology for conveying images to an end user of the communication device 600. In an embodiment where the display 610 is touch-sensitive, a portion or all of the keypad 608 can be presented by way of the display 610 with navigation features.

The display 610 can use touch screen technology to also serve as a user interface for detecting user input. As a touch screen display, the communication device 600 can be adapted to present a user interface having graphical user interface (GUI) elements that can be selected by a user with a touch of a finger. The display 610 can be equipped with capacitive, resistive or other forms of sensing technology to detect how much surface area of a user's finger has been placed on a portion of the touch screen display. This sensing information can be used to control the manipulation of the GUI elements or other functions of the user interface. The display 610 can be an integral part of the housing assembly of the communication device 600 or an independent device communicatively coupled thereto by a tethered wireline interface (such as a cable) or a wireless interface.

The UI 604 can also include an audio system 612 that utilizes audio technology for conveying low volume audio (such as audio heard in proximity of a human ear) and high-volume audio (such as speakerphone for hands free operation). The audio system 612 can further include a microphone for receiving audible signals of an end user. The audio system 612 can also be used for voice recognition applications. The

UI 604 can further include an image sensor 613 such as a charged coupled device (CCD) camera for capturing still or moving images.

The power supply 614 can utilize common power management technologies such as replaceable and rechargeable batteries, supply regulation technologies, and/or charging system technologies for supplying energy to the components of the communication device 600 to facilitate long-range or short-range portable communications. Alternatively, or in combination, the charging system can utilize external power sources such as DC power supplied over a physical interface such as a USB port or other suitable tethering technologies.

The location receiver 616 can utilize location technology such as a global positioning system (GPS) receiver capable of assisted GPS for identifying a location of the communication device 600 based on signals generated by a constellation of GPS satellites, which can be used for facilitating location services such as navigation. The motion sensor 618 can utilize motion sensing technology such as an accelerometer, a gyroscope, or other suitable motion sensing technology to detect motion of the communication device 600 in three-dimensional space. The orientation sensor 620 can utilize orientation sensing technology such as a magnetometer to detect the orientation of the communication device 600 (north, south, west, and east, as well as combined orientations in degrees, minutes, or other suitable orientation metrics).

The communication device 600 can use the transceiver 602 to also determine a proximity to a cellular, Wi-Fi, Bluetooth®, or other wireless access points by sensing techniques such as utilizing a received signal strength indicator (RSSI) and/or signal time of arrival (TOA) or time of flight (TOF) measurements. The controller 606 can utilize computing technologies such as a microprocessor, a digital signal processor (DSP), programmable gate arrays, application specific integrated circuits, and/or a video processor with associated storage memory such as Flash, ROM, RAM, SRAM, DRAM or other storage technologies for executing computer instructions, controlling, and processing data supplied by the aforementioned components of the communication device 600.

Other components not shown in FIG. 6 can be used in one or more embodiments of the subject disclosure. For instance, the communication device 600 can include a slot for adding or removing an identity module such as a Subscriber Identity Module (SIM) card or Universal Integrated Circuit Card (UICC). SIM or UICC cards can be used for identifying subscriber services, executing programs, storing subscriber data, and so on.

The terms “first,” “second,” “third,” and so forth, as used in the claims, unless otherwise clear by context, is for clarity only and does not otherwise indicate or imply any order in time. For instance, “a first determination,” “a second determination,” and “a third determination,” does not indicate or imply that the first determination is to be made before the second determination, or vice versa, etc.

In the subject specification, terms such as “store,” “storage,” “data store,” “data storage,” “database,” and substantially any other information storage component relevant to operation and functionality of a component, refer to “memory components,” or entities embodied in a “memory” or components comprising the memory. It will be appreciated that the memory components described herein can be either volatile memory or nonvolatile memory, or can comprise both volatile and nonvolatile memory, by way of illustration, and not limitation, volatile memory, non-volatile memory, disk storage, and memory storage. Further, nonvolatile memory can be included in read only memory (ROM), programmable ROM (PROM), electrically programmable ROM (EPROM), electrically erasable ROM (EEPROM), or flash memory. Volatile memory can comprise random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Additionally, the disclosed memory components of systems or methods herein are intended to comprise, without being limited to comprising, these and any other suitable types of memory.

Moreover, it will be noted that the disclosed subject matter can be practiced with other computer system configurations, comprising single-processor or multiprocessor computer systems, mini-computing devices, mainframe computers, as well as personal computers, hand-held computing devices (e.g., PDA, phone, smartphone, watch, tablet computers, netbook computers, etc.), microprocessor-based or programmable consumer or industrial electronics, and the like. The illustrated aspects can also be practiced in distributed computing environments where tasks are performed by remote processing devices that are linked through a communications network; however, some if not all aspects of the subject disclosure can be practiced on stand-alone computers. In a distributed computing environment, program modules can be located in both local and remote memory storage devices.

In one or more embodiments, information regarding use of services can be generated including services being accessed, media consumption history, user preferences, and so forth. This information can be obtained by various methods including user input, detecting types of communications (e.g., video content vs. audio content), analysis of content streams, sampling, and so forth. The generating, obtaining and/or monitoring of this information can be responsive to an authorization provided by the user. In one or more embodiments, an analysis of data can be subject to authorization from user(s) associated with the data, such as an opt-in, an opt-out, acknowledgement requirements, notifications, selective authorization based on types of data, and so forth.

Some of the embodiments described herein can also employ artificial intelligence (AI) to facilitate automating one or more features described herein. The embodiments (e.g., in connection with automatically identifying acquired cell sites that provide a maximum value/benefit after addition to an existing communication network) can employ various AI-based schemes for carrying out various embodiments thereof. Moreover, the classifier can be employed to determine a ranking or priority of each cell site of the acquired network. A classifier is a function that maps an input attribute vector, x=(x1, x2, x3, x4 . . . xn), to a confidence that the input belongs to a class, that is, f(x)=confidence (class). Such classification can employ a probabilistic and/or statistical-based analysis (e.g., factoring into the analysis utilities and costs) to determine or infer an action that a user desires to be automatically performed. A support vector machine (SVM) is an example of a classifier that can be employed. The SVM operates by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches comprise, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

As will be readily appreciated, one or more of the embodiments can employ classifiers that are explicitly trained (e.g., via a generic training data) as well as implicitly trained (e.g., via observing UE behavior, operator preferences, historical information, receiving extrinsic information). For example, SVMs can be configured via a learning or training phase within a classifier constructor and feature selection module. Thus, the classifier(s) can be used to automatically learn and perform a number of functions, including but not limited to determining according to predetermined criteria which of the acquired cell sites will benefit a maximum number of subscribers and/or which of the acquired cell sites will add minimum value to the existing communication network coverage, etc.

As used in some contexts in this application, in some embodiments, the terms “component,” “system” and the like are intended to refer to, or comprise, a computer-related entity or an entity related to an operational apparatus with one or more specific functionalities, wherein the entity can be either hardware, a combination of hardware and software, software, or software in execution. As an example, a component may be, but is not limited to being, a process running on a processor, a processor, an object, an executable, a thread of execution, computer-executable instructions, a program, and/or a computer. By way of illustration and not limitation, both an application running on a server and the server can be a component. One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software or firmware that confers at least in part the functionality of the electronic components. While various components have been illustrated as separate components, it will be appreciated that multiple components can be implemented as a single component, or a single component can be implemented as multiple components, without departing from example embodiments.

Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device or computer-readable storage/communications media. For example, computer readable storage media can include, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or”. That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Moreover, terms such as “user equipment,” “mobile station,” “mobile,” “subscriber station,” “access terminal,” “terminal,” “handset,” “mobile device” (and/or terms representing similar terminology) can refer to a wireless device utilized by a subscriber or user of a wireless communication service to receive or convey data, control, voice, video, sound, gaming or substantially any data-stream or signaling-stream. The foregoing terms are utilized interchangeably herein and with reference to the related drawings.

Furthermore, the terms “user,” “subscriber,” “customer,” “consumer” and the like are employed interchangeably throughout, unless context warrants particular distinctions among the terms. It should be appreciated that such terms can refer to human entities or automated components supported through artificial intelligence (e.g., a capacity to make inference based, at least, on complex mathematical formalisms), which can provide simulated vision, sound recognition and so forth.

As employed herein, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components or any combination thereof designed to perform the functions described herein. Processors can exploit nano-scale architectures such as, but not limited to, molecular and quantum-dot based transistors, switches and gates, in order to optimize space usage or enhance performance of user equipment. A processor can also be implemented as a combination of computing processing units.

As used herein, terms such as “data storage,” “data storage,” “database,” and substantially any other information storage component relevant to operation and functionality of a component, refer to “memory components,” or entities embodied in a “memory” or components comprising the memory. It will be appreciated that the memory components or computer-readable storage media, described herein can be either volatile memory or nonvolatile memory or can include both volatile and nonvolatile memory.

What has been described above includes mere examples of various embodiments. It is, of course, not possible to describe every conceivable combination of components or methodologies for purposes of describing these examples, but one of ordinary skill in the art can recognize that many further combinations and permutations of the present embodiments are possible. Accordingly, the embodiments disclosed and/or claimed herein are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

In addition, a flow diagram may include a “start” and/or “continue” indication. The “start” and “continue” indications reflect that the steps presented can optionally be incorporated in or otherwise used in conjunction with other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

As may also be used herein, the term(s) “operably coupled to”, “coupled to”, and/or “coupling” includes direct coupling between items and/or indirect coupling between items via one or more intervening items. Such items and intervening items include, but are not limited to, junctions, communication paths, components, circuit elements, circuits, functional blocks, and/or devices. As an example of indirect coupling, a signal conveyed from a first item to a second item may be modified by one or more intervening items by modifying the form, nature or format of information in a signal, while one or more elements of the information in the signal are nevertheless conveyed in a manner than can be recognized by the second item. In a further example of indirect coupling, an action in a first item can cause a reaction on the second item, as a result of actions and/or reactions in one or more intervening items.

Although specific embodiments have been illustrated and described herein, it should be appreciated that any arrangement which achieves the same or similar purpose may be substituted for the embodiments described or shown by the subject disclosure. The subject disclosure is intended to cover any and all adaptations or variations of various embodiments. Combinations of the above embodiments, and other embodiments not specifically described herein, can be used in the subject disclosure. For instance, one or more features from one or more embodiments can be combined with one or more features of one or more other embodiments. In one or more embodiments, features that are positively recited can also be negatively recited and excluded from the embodiment with or without replacement by another structural and/or functional feature. The steps or functions described with respect to the embodiments of the subject disclosure can be performed in any order. The steps or functions described with respect to the embodiments of the subject disclosure can be performed alone or in combination with other steps or functions of the subject disclosure, as well as from other embodiments or from other steps that have not been described in the subject disclosure. Further, more than or less than all of the features described with respect to an embodiment can also be utilized.

What is claimed is:

1. A device, comprising:
a processing system including a processor; and
a memory that stores executable instructions that, when executed by the processing system, facilitate performance of operations, the operations comprising:
determining a performance event of a user;
detecting a difference between current biometric information of the user and target biometric information of the user exceeding a threshold at the performance event to identify a biometric deficiency;
detecting an absence of a desired environmental component for the user at the performance event;
selecting a remediation experience responsive to the detecting the biometric deficiency and the detecting the absence of the desired environmental component;
facilitating presentation of the remediation experience to the user at the performance event to generate a modified performance by the user;
monitoring the modified performance by the user responsive to the facilitating the presentation of the remediation experience to the user at the performance event;
adjusting the presentation of the remediation experience to the user at the performance event according to the monitoring the performance of the user;
detecting a termination of the performance event; and
adjusting the presentation of the remediation experience to the user responsive to detecting the termination of the performance event.

2. The device of claim 1, wherein the operations further comprise determining the current biometric information of the user at the performance event.

3. The device of claim 2, wherein the determining the current biometric information of the user includes accessing a sensor worn by the user.

4. The device of claim 1, wherein the determining the performance event of the user includes accessing location information, video information, audio information, calendar information, or a combination thereof, associated with the user.

5. The device of claim 1, wherein the operations further comprise determining the desired biometric information of the user at the performance event.

6. The device of claim 5, wherein the determining the desired biometric information of the user at the performance includes accessing historical biometric information data associated with the user.

7. The device of claim 1, wherein the operations further comprise determining a desired environmental component for the user at the performance event.

8. The device of claim 1, wherein the operations further comprise:
receiving information from a supervisory system associated with the performance event; and
adjusting the presentation of the remediation experience to the user at the performance event according to the information from the supervisory system.

9. The device of claim 1, wherein the presenting the remediation experience includes generating audio for the user to experience at the performance event.

10. The device of claim 1, wherein the presenting the remediation experience includes generating video for the user to experience at the performance event.

11. The device of claim 1, wherein the presenting the remediation experience includes generating haptic feedback for the user to experience at the performance event.

12. The device of claim 1, wherein the presentation of the remediation experience enhances a perceived reality for the user at the performance event.

13. The device of claim 1, wherein the presentation of the remediation experience diminishes a perceived reality for the user at the performance event.

14. A non-transitory machine-readable medium, comprising executable instructions that, when executed by a processing system including a processor, facilitate performance of operations, the operations comprising:
detecting a difference between current biometric information of a user and target biometric information of the user exceeding a threshold at a performance event of the user to identify a biometric deficiency;
detecting an absence of a desired environmental component for the user at the performance event;
selecting a remediation experience responsive to the detecting the biometric deficiency or the detecting the absence of the desired environmental component;
facilitating presentation of the remediation experience to the user at the performance event to generate a modified performance by the user;
monitoring the modified performance by the user responsive to the facilitating the presentation of the remediation experience to the user at the performance event;
adjusting the presentation of the remediation experience to the user at the performance event according to the monitoring the performance of the user; and
adjusting the presentation of the remediation experience to the user at a termination of the performance event.

15. The non-transitory machine-readable medium of claim 14, wherein the operations further comprise determining the performance event of the user by accessing location information, video information, audio information, calendar information, or a combination thereof, associated with the user.

16. The non-transitory machine-readable medium of claim 14, wherein the operations further comprise determining the current biometric information of the user at the performance event by accessing a sensor worn by the user.

17. The non-transitory machine-readable medium of claim 14, wherein the operations further comprise determining a desired environmental component for the user at the performance event according to a user preference.

18. A method, comprising:
detecting, by a processing system including a processor, a difference between current biometric information of a user and target biometric information of the user exceeding a threshold at a performance event of the user to identify a biometric deficiency;
detecting, by the processing system, an absence of a desired environmental component for the user at the performance event;
selecting, by the processing system, a remediation experience responsive to detecting the biometric deficiency or the detecting the absence of the desired environmental component;
facilitating, by the processing system, presentation of the remediation experience to the user at the performance event to generate a modified performance by the user; and
adjusting, by the processing system, the presentation of the remediation experience to the user at a termination of the performance event.

19. The method of claim 18, further comprising:
monitoring, by the processing system, the modified performance by the user responsive to the facilitating the presentation of the remediation experience to the user at the performance event; and
adjusting, by the processing system, the presentation of the remediation experience to the user at the performance event according to the monitoring the performance of the user.

20. The method of claim 18, further comprising determining, by the processing system, the performance event of the user by accessing location information, video information, audio information, calendar information, or a combination thereof, associated with the user.
